(19) United States
(12) Patent Application Publication
(10) Pub. No.: US 2025/0160641 A1
(43) Pub. Date: May 22, 2025

(54) METHOD FOR AUTOMATICALLY GENERATING FLUORESCEIN ANGIOGRAPHY IMAGES BASED ON NONINVASIVE FUNDUS IMAGES

(71) Applicant: Zhongshan Ophthalmic Center Sun Yat-sen University, Guangzhou (CN)

(72) Inventors: Mingguang He, Guangzhou (CN); Danli Shi, Guangzhou (CN)

(21) Appl. No.: 19/028,612

(22) Filed: Jan. 17, 2025

Related U.S. Application Data
(63) Continuation of application No. PCT/CN2022/133742, filed on Nov. 23, 2022.

(30) Foreign Application Priority Data
Aug. 2, 2022 (CN) 2022109207789

(51) Int. Cl. A61B 3/12; A61B 3/14

(52) U.S. Cl. CPC A61B 3/1241; A61B 3/14

(57) ABSTRACT

A method for automatically generating fluorescein angiography images based on noninvasive fundus images, using noninvasive fundus images and matched multi-timepoint fluorescein angiography images as training data to train and generate a conditional generative adversarial network, and to construct the fluorescein angiography images based on the noninvasive fundus images. After the fluorescein angiography images are constructed, it can take noninvasive fundus images as input to generate corresponding early, middle, and late phase fluorescein angiography images. The generated fluorescein angiography images can clearly display the retinal structure and the fluorescence characteristics of various lesions. The method can reduce the reliance on fluorescein angiography, an invasive diagnostic technology with significant risk of side effects, and enhance the ability to diagnose eye diseases.

### TECHNICAL FIELD

The disclosure relates to the field of image model construction technologies, and particularly to a method for automatically generating fluorescein angiography images based on noninvasive fundus images.

### BACKGROUND

Retinal fluorescein angiography is an important routine examination method in ophthalmology and is a gold standard for examining retinal vascular diseases and revealing leakage. However, fluorescein angiography requires the injection of contrast agents into the vein, which is an invasive examination and is limited by adverse reactions such as contrast agent allergies.

Noninvasive fundus photography is the most commonly used method for retinal examination, with advantages of being non-invasive and fast, but it cannot dynamically and clearly display abnormal structural changes of the retina, and many characteristic lesions of the fundus cannot be presented on noninvasive fundus images.

Artificial intelligence technology is used to train a conditional generative network with fluorescein angiography images as labels to generate high-quality fluorescein angiography images based on noninvasive fundus images can complement the advantages of the two examination modes of the fluorescein angiography and the noninvasive fundus photography, and is expected to improve the early and accurate detection of ophthalmic diseases and bypass the need for fluorescein dye injection.

### SUMMARY

The disclosure provides a method for automatically generating fluorescein angiography images based on noninvasive fundus images, the method uses the noninvasive fundus images as input to automatically generate fluorescein angiography images that include various lesion features.

To achieve the above purpose, the disclosure provides the following technical solutions as follows.

S1: the noninvasive fundus images and fluorescein angiography images at different angiography periods for an eye are collected to ensure that retinal structures of the noninvasive fundus images and retinal structures of the fluorescein angiography images are in eye-to-eye correspondence.

S2: the fluorescein angiography images are used as a gold standard, a generation model is trained, the noninvasive fundus images are input, and then the fluorescein angiography images are constructed based on the noninvasive fundus images.

In an embodiment, in the S1, the noninvasive fundus images include a regular view noninvasive fundus image to an ultra-wide field image, with degrees ranging from 30 to 200 degrees.

In an embodiment, in the S1, the noninvasive fundus images are obtained by non-invasive fundus imaging, and the non-invasive fundus imaging includes multi-spectral imaging, infra-red imaging, optical coherence tomography, and optical coherence tomography angiography.

In an embodiment, in the S1, angiography images are not limited to the fluorescein angiography images, the angiography images include indocyanine green angiography or other kinds of dye injection.

In an embodiment, in the S2, the conditional generative adversarial network includes a generator and a discriminator.

In an embodiment, in the S2, the generation model includes conditional generative adversarial networks, diffusion model, and diffusion transformer. The conditional generative adversarial network is an example implementation.

The noninvasive fundus images are input, and the generator is used to generate the fluorescein angiography images.

Real fluorescein angiography images are used as the gold standard to train the model and to distinguish the difference between the generated fluorescein angiography images and the gold standard by the discriminator. This process creates feedback between the generator and the discriminator, which allows for the continuous updating of the generator's network parameters until a best-game balance is achieved. The balance indicates that the outputted generated fluorescein angiography images are closest to the real fluorescein angiography images. At this point, the generator is extracted for use when the model is constructed.

Other methods that improve the retinal structure and lesion generation quality are added, including high-frequency component loss function, using lesion segmentation and thresholded lesion masks as guidance.

Compared to the related art, the beneficial effects of the disclosure are as follows.

The disclosure includes analyzing, detecting, and measuring lesions in the fluorescein angiography images, and specifically includes that: the fluorescein angiography images is used to identify the lesions in blood vessels, the macula, the optic disc, and other regions. The lesions include but not limited to microaneurysms, retinal non-perfusion areas, retinal neovascularization, choroidal neovascularization, macular edema, retinal edema, abnormal retinal pigment epithelium leakage, and dilated retinal capillary leakage. The fluorescein angiography images generated by the method of the disclosure can be used for imaging diagnosis and quantitative detection and measurement of the lesions.

The disclosure can use the fluorescein angiography images generated by the method of the disclosure for independent or combined diagnosis of fundus diseases with the noninvasive fundus images or another imaging modalities.

The disclosure performs a lesion detection and an extraction on the real fluorescein angiography images, and uses the detected lesion feature images as labels (the gold standard) to train conditional generative adversarial network, thereby directly generating retinal lesions. The generated retinal lesions can be analyzed and measured to assist in disease diagnosis.

### BRIEF DESCRIPTION OF DRAWINGS

FIG. 1 illustrates a flowchart of the disclosure.

FIG. 2 illustrates a training flowchart of a generative adversarial network according to an embodiment of the disclosure.

FIG. 3 illustrates a schematic diagram comparing a display of disease lesions in a real fluorescein angiography image (i.e., real fluorescein angiography image) and a generated fluorescein angiography image according to an embodiment of the disclosure, including an approximately normal retina, choroidal neovascularization leakage, and optic disc edema.

FIG. 4 illustrates a schematic diagram comparing a display of disease lesions in a real fluorescein angiography image and a generated fluorescein angiography image according to an embodiment of the disclosure, including macular capillary telangiectasia leakage, and severe macular edema.

FIG. 5 illustrates a schematic diagram comparing a display of disease lesions in a real fluorescein angiography image and a generated fluorescein angiography image according to an embodiment of the disclosure, including significant leakage of retinal neovascularization, and extensive retinal edema.

FIG. 6 illustrates a schematic diagram comparing a display of disease lesions in a real fluorescein angiography image and a generated fluorescein angiography image according to an embodiment of the disclosure, including small and large areas of retinal non-perfusion.

FIG. 7 illustrates a schematic diagram comparing a display, a detection, and a segmentation of microaneurysms in the real fluorescein angiography image and the generated fluorescein angiography image of the embodiment of the disclosure.

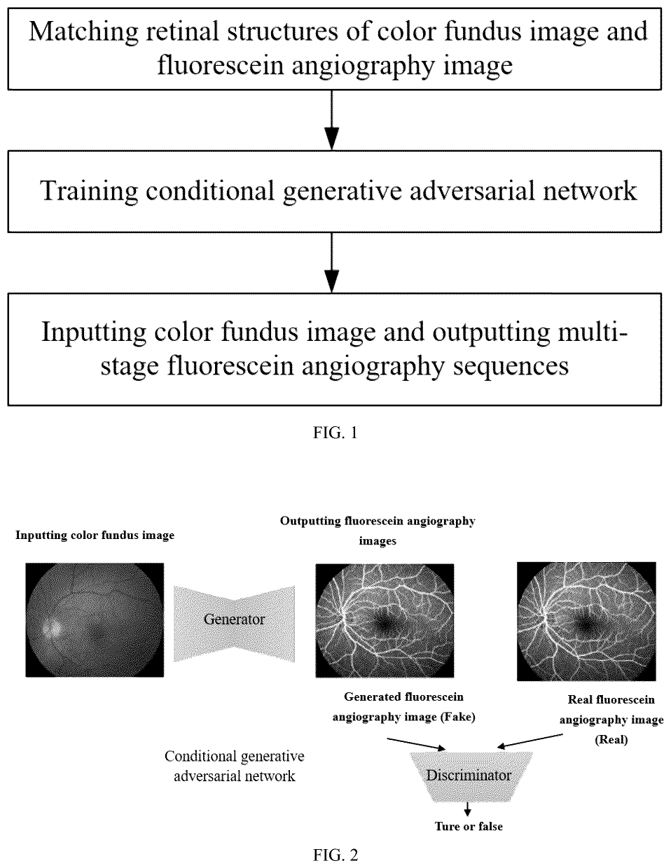

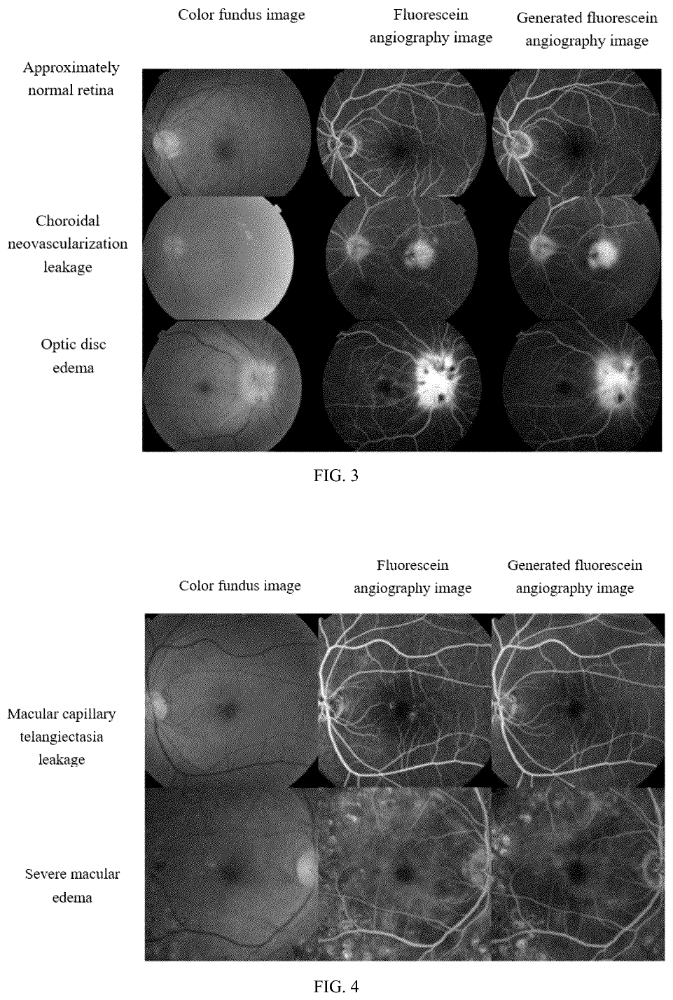

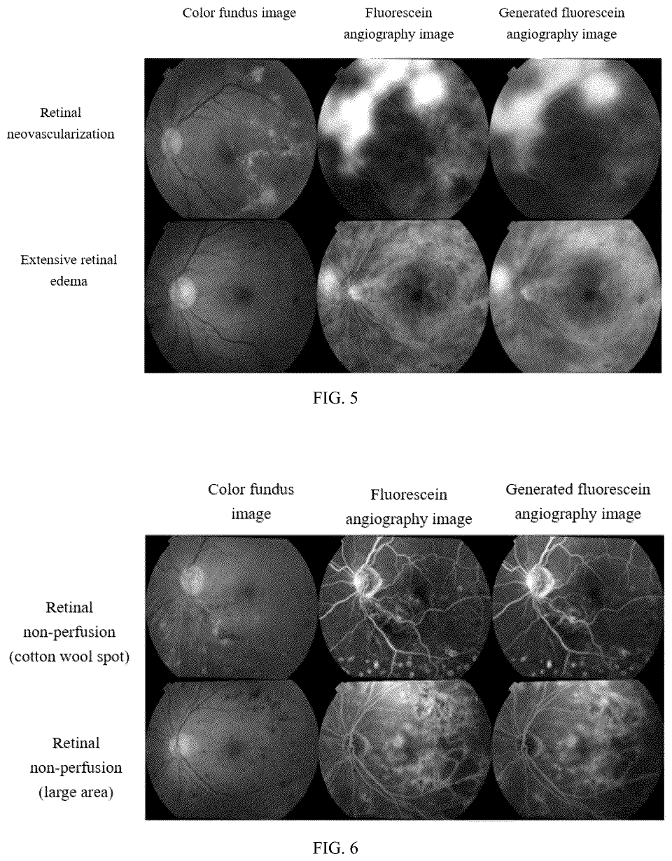

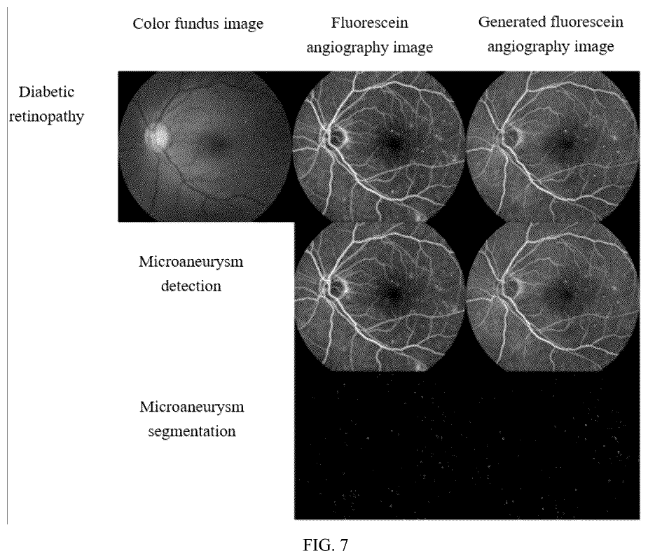

### DETAILED DESCRIPTION OF EMBODIMENTS

To achieve the purpose, features, and advantages of the disclosure more obvious and understandable, the specific embodiments of the disclosure will be described in detail below in conjunction with the attached drawings. Several embodiments of the disclosure are shown in the attached drawings. However, the disclosure can be implemented in many different forms and is not limited to the embodiments described herein. On the contrary, the purpose of providing the embodiments is to make the disclosure more thorough and comprehensive.

A method for automatically generating fluorescein angiography images based on noninvasive fundus images includes steps as follows.

S1: the noninvasive fundus images and fluorescein angiography images at different angiography periods for an eye are collected to ensure that retinal structures of the noninvasive fundus images and retinal structures of the fluorescein angiography images are in eye-to-eye correspondence.

S2: the fluorescein angiography images are used as a gold standard, a generation model is trained, the noninvasive fundus images are input, and then the fluorescein angiography images are constructed based on the noninvasive fundus images.

In an embodiment, in the S1, the noninvasive fundus images include a regular view noninvasive fundus image to an ultra-wide field image, with degrees ranging from 30 to 200 degrees.

In an embodiment, in the S1, the noninvasive fundus images are obtained by non-invasive fundus imaging, and the non-invasive fundus imaging includes multi-spectral imaging, infra-red imaging, optical coherence tomography, and optical coherence tomography angiography.

In an embodiment, in the S1, angiography images are not limited to the fluorescein angiography images, the angiography images include indocyanine green angiography or other kinds of dye injection.

In an embodiment, in the S2, the conditional generative adversarial network includes a generator and a discriminator.

In an embodiment, in the S2, the generation model includes conditional generative adversarial networks, diffusion model, and diffusion transformer. The conditional generative adversarial network is an example implementation.

The noninvasive fundus images are input, and the generator is used to generate the fluorescein angiography images.

Real fluorescein angiography images are used as the gold standard to train the model and to distinguish difference between the generated fluorescein angiography images and the gold standard by the discriminator. This process creates feedback between the generator and the discriminator, which allows for the continuous updating of the generator's network parameters until a best game balance is achieved. The balance indicates that the outputted generated fluorescein angiography images are closest to the real fluorescein angiography images. At this point, the generator is extracted for use when the model is constructed.

The embodiments described above only express several embodiments of the disclosure, which are described in a more specific and detailed manner, but this should not be understood as limiting the scope of the disclosure. It should be noted that for those skilled in the art, without departing from the concept of the disclosure, various modifications and improvements can be made, all of which fall within the scope of protection of the disclosure. Therefore, the scope of protection of the disclosure should be determined by the appended claims.

What is claimed is:

1. A method for automatically generating fluorescein angiography images based on noninvasive fundus images, comprising steps as follows:
S1: collecting the noninvasive fundus images and fluorescein angiography images at different angiography periods for an eye, ensuring that retinal structures of the noninvasive fundus images and retinal structures of the fluorescein angiography images are in eye-to-eye correspondence; and
S2: using fluorescein angiography images as a gold standard, training a conditional generative adversarial network, inputting the noninvasive fundus images, and then constructing the fluorescein angiography images based on the noninvasive fundus images.

2. The method as claimed in claim 1, wherein the noninvasive fundus images in the step S1 comprise a regular view noninvasive fundus image to an ultra-wide field fundus image, and the fluorescein angiography images comprise a retinal vascular fluorescein angiography image and a choroidal vascular fluorescein angiography image.

3. The method as claimed in claim 1, wherein the conditional generative adversarial network in the step S2 comprise a generator and a discriminator;
the step S2 further comprises:
inputting the noninvasive fundus images, and using the generator to generate the fluorescein angiography images; and
using real fluorescein angiography images as the gold standard, distinguishing difference between the generated fluorescein angiography images and the gold standard by the discriminator, thereby creating feedback between the generator and the discriminator to continuously update network parameters of the generator until a best game balance is achieved, meaning that the outputted generated fluorescein angiography images are closest to the real fluorescein angiography images; at this point, extracting the generator for use when the model is constructed.
